(19) United States
(12) Patent Application Publication
(10) Pub. No.: US 2024/0042396 A1
(43) Pub. Date: Feb. 8, 2024

(54) Polymeric Membrane

(71) Applicant: 3M INNOVATIVE PROPERTIES COMPANY, St. Paul, MN (US)

(72) Inventors: Oliver Schuster, Gevelsberg (DE); Neelakandan Chandrasekaran, Plymouth, MN (US); Detlef Krieter, Wurzburg (DE); Stefan Weuster, Remscheid (DE); Holger Gottschalk, Wuppertal (DE)

(21) Appl. No.: 18/266,484

(22) PCT Filed: Dec. 6, 2021
(86) PCT No.: PCT/IB2021/061375

§ 371 Date: Jun. 9, 2023

Related U.S. Application Data
(60) Provisional application No. 63/279,859, filed on Nov. 16, 2021.
(60) Provisional application No. 63/123,837, filed on Dec. 10, 2020.

(51) Int. Cl. B01D 71/68; B01D 69/08; B01D 67/00; B01D 71/74; B01D 61/24

(52) U.S. Cl. CPC B01D 71/68; B01D 69/08; B01D 67/0088; B01D 71/74; B01D 61/246

(57) ABSTRACT

A polymeric membrane. The membrane can include a polymeric membrane made from a polymer selected from an aromatic sulfone polymer, polyamide, cellulose, cellulose acetate, polymethylmethacrylate, polyvinylalcohol, and polyacrylnitril, wherein the polymeric membrane has a major surface; a stilbenoid, isoflavone or flavone coated on the major surface of the polymeric membrane.

### FIELD

The present disclosure relates to microporous membranes. In addition, the present disclosure relates to a process for producing such membranes. The present disclosure further relates to use of such membranes for filtration and purification of liquid media.

### BACKGROUND

Polymer membranes are employed in a very wide range of different industrial, pharmaceutical or medical applications for precision filtration. In these applications, membrane separation processes are gaining in importance, as these processes offer the advantage that the substances to be separated are not thermally burdened or even damaged. Ultrafiltration membranes can be employed for the removal or separation of macromolecules. Numerous further applications of membrane separation processes are known from the beverages industry, biotechnology, water treatment or sewage technology. Such membranes are generally classified according to their retention capacity, i.e. according to their capacity for retaining particles or molecules of a certain size, or with respect to the size of the effective pores, i.e. the size of the pores that determine the separation behavior. Ultrafiltration membranes thereby cover the size range of the pores determining the separation behavior between roughly 0.01 and approx. 0.1 μm, so that particles or molecules with a size in the range larger than 20 000 or larger than approx. 200 000 Daltons can be retained. There is a need for better polymer membranes.

### SUMMARY

Thus, in one aspect, the present disclosure provides a membrane comprising: a polymeric membrane made from a polymer selected from an aromatic sulfone polymer, polyamide, cellulose, cellulose acetate, polymethylmethacrylate, polyvinylalcohol, and polyacrylnitril, wherein the polymeric membrane has a major surface; a stilbenoid, isoflavone or flavone coated on the major surface of the polymeric membrane.

In another aspect, the present disclosure provides a method, the method comprising: forming a polymeric membrane from an aromatic sulfone polymer; and coating a stilbenoid, isoflavone or flavone to the hollow fiber membrane.

In another aspect, the present disclosure provides a use of the polymeric membrane of present disclosure for filtration of liquids.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

The present disclosure provides a membrane comprising a polymeric membrane having a major surface and a wall having a wall thickness. In some embodiments, the polymeric membrane can be a hollow membrane and the hollow membrane may have a continuous hollow lumen, which extends from one end to the other end of the fiber, an outer surface facing outwards, which forms an outer side of the fiber; an inner surface facing towards the hollow lumen, which defines the limits of the continuous hollow lumen; and an intermediate wall having a wall thickness. In some embodiments of the hollow membrane, the major surface can be the inner surface. In some embodiments of the hollow membrane, the major surface can be the inner surface. The polymeric membrane can include a stilbenoid, isoflavone or flavone coated on the major surface of the polymeric membrane. In some cases, the stilbenoid, isoflavone or flavone may form a layer and may at least partially cover the major surface. In some embodiments, the stilbenoid, isoflavone or flavone may cover more than 50%, 60%, 70%, 75%, 80%, 85%, 90%, 95%, 96%, 97%, 98%, or 99% major surface of the polymeric membrane. In some embodiments, the stilbenoid, isoflavone or flavone may cover 100% major surface of the polymeric membrane. In some embodiments, the wall can comprise a plurality of pores and the stilbenoid, isoflavone or flavone can be coated on the surface of at least some of the plurality of pores. In some embodiments, the stilbenoid, isoflavone or flavone may cover more than 50%, 60%, 70%, 75%, 80%, 85%, 90%, 95%, 96%, 97%, 98%, or 99% of the surface of at least some of the plurality of pores. In some embodiments, the stilbenoid, isoflavone or flavone may cover 100% of the surface of at least some of the plurality of pores. In some embodiments, the stilbenoid, isoflavone or flavone can be coated on the surface of more than 50%, 60%, 70%, 75%, 80%, 85%, 90%, 95%, 96%, 97%, 98%, or 99% of the plurality of pores. In some embodiments, the stilbenoid, isoflavone or flavone may cover the surface of all of the plurality of pores. In some embodiments, the stilbenoid, isoflavone or flavone may be coated on and cover at least a part of the outer surface. In some embodiments, the stilbenoid, isoflavone or flavone may cover more than 50%, 60%, 70%, 75%, 80%, 85%, 90%, 95%, 96%, 97%, 98%, or 99% of the outer surface. In some embodiments, the stilbenoid, isoflavone or flavone may cover 100% of the outer surface. In some embodiments, the polymeric membrane can be a flat sheet membrane having two major surfaces: the first major surface and the second major surface opposite the first major surface. In some of these embodiments, the stilbenoid, isoflavone or flavone may at least partially cover both the first major surface and the second major surface. In some of these embodiments, the stilbenoid, isoflavone or flavone may cover more than 50%, 60%, 70%, 75%, 80%, 85%, 90%, 95%, 96%, 97%, 98%, or 99% both the first major surface and the second major surface.

The wall thickness, (in the embodiments of hollow fiber membrane, the wall thickness can be measured between the outer surface and the inner surface of the hollow fiber membrane), can be in the range of from 20 to 500 μm, from 140 to 400 μm, from 150 to 380 μm, or from 160 to 380 μm. In some embodiments, in order to achieve a desirable flow through the lumina of the hollow-fiber membranes according to the present disclosure, particularly, a favourable pressure drop, it is preferred that the inside diameter of the hollow-fiber membranes as described herein is in the range of from 100 to 2000 μm, from 700 to 2000 μm, from 800 to 1800 μm, or from 900 to 1600 μm. Wall thicknesses and diameters (in the embodiments of hollow fiber membrane, inner or lumen diameter, and outer diameter) of the membranes as described herein are also determined by means of conventional examination methods, such as using scanning or transmission electron micrographs (SEM or TEM, respectively), for example with a magnification of 400:1.

The polymeric membranes according to the present disclosure can be made by methods disclosed in WO 2019/229667 A1 (Malek et al.), which is incorporated herein by reference in their entirety into this disclosure. In some embodiments, the polymeric membranes can be made from a homogeneous spinning solution of a polymer component and a solvent system. The polymer component thereby comprises a polymer selected from an aromatic sulfone polymer, polyamide, cellulose, cellulose acetate, polymethylmethacrylate, polyvinylalcohol, and polyacrylnitril. The polymer component can further comprise at least one hydrophilic polymer. The flat sheet membranes and methods for their production are described e.g. in EP 0361 085 B1.

According to the present disclosure, the concentration of the sulfone polymer in the spinning solution is preferably in the range of from 17 to 27 wt. %. Below a concentration of 17 wt. %, disadvantages may arise in particular with respect to the mechanical stability of the hollow-fiber membranes obtained. On the other hand, membranes obtained from spinning solutions with more than 27 wt. % of the sulfone polymer may exhibit an excessively dense structure and insufficient permeability. The spinning solution preferably contains 20 to 25 wt. % of the hydrophobic aromatic sulfone polymer. The sulfone polymer can also contain additives such as antioxidants, nucleating agents, UV absorbers, etc. to selectively modify the properties of the membranes.

Advantageous hydrophobic aromatic sulfone polymers from which the membrane according to the present disclosure is composed or which are employed in the method according to the invention are polysulfone, polyether sulfone, polyphenylene sulfone or polyaryl ether sulfone. Preferably, the hydrophobic aromatic sulfone polymer is a polysulfone or a polyether sulfone with the repeating molecular units shown in the following formulae (I) and (II):

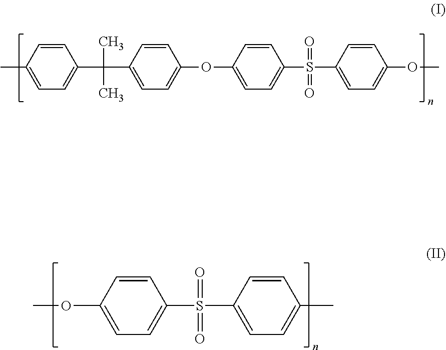

Long-chain polymers are advantageously employed as the at least one hydrophilic polymer that on the one hand exhibit a compatibility with the hydrophobic aromatic sulfone polymer and have repeating polymer units that in themselves are hydrophilic. A hydrophilic polymer with a mean molecular weight M_{W} of more than 10 000 Daltons, preferably of more than 20 000 Daltons, more preferably of more than 30 000 Daltons, is preferably employed. The hydrophilic polymer is preferably polyvinylpyrrolidone, polyethylene glycol, polyvinyl alcohol, polyglycol monoester, a polysorbitate such as polyoxyethylene sorbitan monooleate, carboxymethylcellulose or a modification or copolymer of these polymers. Polyvinylpyrrolidone and polyethylene glycol are particularly preferred.

Within the context of the present disclosure, the at least one hydrophilic polymer can also comprise mixtures of different hydrophilic polymers. The hydrophilic polymer can, for example, be a mixture of chemically different hydrophilic polymers or of hydrophilic polymers with different molecular weights, e.g. a mixture of polymers whose molecular weight differs by a factor of 5 or more. Preferably, the at least one hydrophilic polymer comprises a mixture of polyvinylpyrrolidone or polyethylene glycol with a hydrophilically modified aromatic sulfone polymer. It is also preferred that the hydrophilically modified aromatic sulfone polymer is a sulfonated aromatic sulfone polymer, in particular a sulfonated modification of the hydrophobic aromatic sulfone polymer employed in the membrane and in the method according to the present disclosure. Mixtures of polyether sulfone, sulfonated polyether sulfone and polyvinylpyrrolidone can be particularly advantageously employed. As a result of the presence of a hydrophilically modified aromatic sulfone polymer, hollow-fiber membranes with particularly stable hydrophilic properties in the application are obtained.

The polymeric membrane made from the aromatic sulfone polymer can then be coated a stilbenoid, isoflavone or flavone on its major surface. The stilbenoid, isoflavone or flavone can be dissolved in a solvent to form a coating solution. The solvent can be selected from the group consisting of ethanol and isopropanol. In some embodiments, the coating solution can be prepared by dissolving stilbenoid, isoflavone or flavone in the solvent less than 1 wt %, less than 0.9 wt %, less than 0.8 wt %, less than 0.7%, less than 0.6%, or less than 0.5% wt % at room temperature. In some embodiments, the coating solution can be prepared by dissolving stilbenoid, isoflavone or flavone in the solvent at 0.8 wt %, 0.7%, 0.6%, 0.5% wt %, or 0.4 wt %. The polymeric membrane can be immersed with the coating solution. After the polymeric membrane is immersed with the coating solution and is incubated for certain time, for example, 10 minutes, the coating solution can be drained from the polymeric membrane. After the polymeric membrane is immersed with the coating solution and is incubated for certain time, for example, 10 minutes, the coating solution is drained from the polymeric membrane. To dry the polymeric membrane, the polymeric membrane can be connected to nitrogen source applying a gentle gas stream through the membranes evaporating the solvent and leaving the stilbenoid, isoflavone or flavone coated on the major surface of the polymeric membrane.

In the embodiments of hollow fiber membrane, the coating solution can be flowed into the lumen of the hollow fiber membrane. Due to the very good wettability of the solvent on the aromatic sulfone polymer fibers, the coating solution can cover the membrane wall and also sip into the extra-capillary volume. After the lumen is completely filled with the coating solution and is incubated for certain time, for example, 10 minutes, the coating solution can be drained from the hollow fiber membrane. After this step, the hollow fiber membrane can be still completely soaked with the coating solution due to capillary forces.

The (iso)flavone used in the current application can include those disclosed in U.S. Pat. No. 8,883,010 B2 (Chandrasekaran et al.), for example, a flavone, isoflavone or combination thereof. The exemplary (iso)flavone can be an isolated, naturally occurring isoflavone(s), synthesized isoflavone(s), or a combination thereof. In the exemplary embodiment, the flavone or isoflavone may be an hydroxy(iso)flavone (i.e., a hydroxyflavone or hydroxyisoflavone), having at least one hydroxyl group, such as a polyphenol, i.e., a molecule having at least two phenol groups. Although not fully understood, it is believed that the phenol group(s) in a hydroxy(iso)flavone contribute to the antioxidant properties of the hydroxy(iso)flavone molecule and aid in the retention of the molecule within the matrix.

In one embodiment, the (iso)flavone can include includes at least one of a hydroxyflavone and a hydroxyisoflavone. The hydroxyflavone or hydroxyisoflavone can be a mono-, di-, tri, or tetra-hydroxyisoflavone (i.e., 1, 2, 3, or 4 of the hydrogens of the flavones or isoflavone molecule are substituted with a hydroxyl group), e.g., a trihydroxyisoflavone. The hydroxyflavone or hydroxyisoflavone may further include one or more additional substituents, such as an alkoxy and/or glucose moiety. In one embodiment, the phytochemical includes a hydroxyisoflavone, such as a mono-, di- or trihydroxy isoflavone.

In one embodiment, the hydroxyisoflavone is a substituted derivative of isoflavone, being related to the isoflavone molecule by the replacement of one, two, three, or four hydrogen atoms with hydroxyl groups. In some embodiments, the isoflavone structure may be additionally substituted with one or more alkoxy group, e.g., methoxy or ethoxy groups.

Exemplary hydroxyisoflavones can be selected from:
genistein (5,7-dihydroxy-3-(4-hydroxyphenyl)chromen-4-one, also known as 4′,5,7-trihydroxvisoflavone) with the following structure:

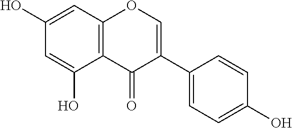 [SMILES: O=c1c(-c2ccc(O)cc2)coc2cc(O)cc(O)c12]

daidzein (7-hydroxy-3-(4-hydroxyphenyl) chromen-4-one (IUPAC), or 4′,7-dihydroxyisoflavone), with the following structure:

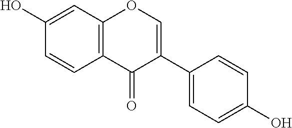 [SMILES: O=c1c(-c2ccc(O)cc2)coc2cc(O)ccc12]

glycitein (7-hydroxy-3-(4-hydroxyphenyl)-6-methoxy-4-chromenone (IUPAC), or 4′,7-dihydroxy-6-methoxyisoflavone),
prunetin (5-hydroxy-3-(4-hydroxyphenyl)-7-methoxychromen-4-one, or 4′,5-dihydroxy-7-methoxyisoflavone),
biochanin A (5,7-dihydroxy-3-(4-methoxyphenyl)chromen-4-one, or 5,7-dihydroxy-4′-methoxyisoflavone),
orobol (3-(3,4-dihydroxyphenyl)-5,7-dihydroxychromen-4-one, or 3′,4′,5,7-tetrahydroxyisoflavone),
santal (7-methoxy-5,3′,4′-trihydroxyisoflavone),
pratensein (5,7-dihydroxy-3-(3-hydroxy-4-methoxyphenyl)chromen-4-one, or 4′-methoxy-3′,5,7-trihydroxyisoflavone),
formononetin (7-hydroxy-3-(4-methoxyphenyl)chromen-4-one, or 7-hydroxy-4′-methoxyisoflavone),
and glucosides, β-glycosides, and a koxy substituted derivatives thereof and combinations thereof.

In some embodiments, the isoflavone may include at least one of the group consisting of genistein and daidzein. In some embodiments, the isoflavone may comprise a mixture of two or more isoflavones.

In some embodiments, the isoflavone may include Genistein. Genistein is of particular interest as a hydroxyisoflavone. It has a molecular weight of 270 g/mol and melts at 306° C. it can reduce oxidative stress and reduce the concentrations of pro-inflammatory cytokines without being toxic or activating the platelet adhesion process.

Stilbenoid used in the current application can include Aglycones, for example, piceatannol, pinosylvin, pterostilbene, resveratrol, gnetol, oxyresveratol, and Glycosides, for example, astringin and piceid. Stilbenoids are hydroxylated derivatives of stilbene. They have a C₆—C₂—C₆ structure. In biochemical terms, they belong to the family of phenylpropanoids.

The polymeric membrane made according to the method of the present disclosure can provide a homogeneous/uniform distribution of the stilbenoid, isoflavone or flavone on the major surface (in some embodiments, the entire major surface) of the polymeric membrane even at lower loading levels (up to 10 wt %). Therefore, the polymeric membrane of the present disclosure can provide a high surface concentration and bulk density of the stilbenoid, isoflavone or flavone on the major surface of the polymeric membrane, even with a low loading concentration, compared the polymeric membrane made by the mixture of the polymer and flavone.

The polymeric membrane of the present disclosure using stilbenoid or flavone, for example, genistein as the active coating, are able to reduce dialysis induced oxidative stress (DIOS) and membrane induced inflammation (MII) by reducing reactive oxygen levels and levels of some cytokines. Cytokines are a family of proteins that are involved in numerous immunological functions including the production and control of other cytokines. They play an important role in the regulation of hematopoiesis, mediating the differentiation and proliferation of diverse type of cells. For example, it has been identified that endotoxins (such as bacterial components) from dialysate induce secretion of IL-1β from neutrophils, which causes fever and low blood pressure during hemodialysis. IL-1β and TNF-α are known for their autocrine (i.e., induce/regulate its own secretion) and paracrine signaling (induce/regulate other cytokine secretion) functions. Clinically, it has been demonstrated that serum concentrations of IL-1β and TNF-α raise several folds during hemodialysis in a manner dependent on the choice of the membrane. Although a polymeric membrane surface could induce cytokine secretion due to direct contact of PBMC with membrane and endotoxin from dialysate, complement-mediated cytokine secretion has been generally accepted as the common mechanism by which hemodialysis membranes induce inflammation In the case of hemodialysis, the alternate pathway of complement activation leads to the formation of complement fragments such as C3b, which coats the membrane surface by adsorbing to the membrane surface. C3b molecules together with other soluble complement fragments such as C3a and C5a subsequently stimulate the PBMC triggering the enhanced secretion of pro-inflammatory cytokines. DIOS is initiated when the excess production of oxygen radicals overpowers the natural antioxidant defense mechanisms of the body. MII causes undesirable immune responses induced by higher concentration of pro-inflammatory cytokines such as interleukin-1β (IL-1β), interleukin (IL-6) and tumor necrosis factor-R (TNF-R) in blood. The bio-incompatibility of the polymeric membranes has been implicated as the primary source of generation of excessive reactive oxygen species (ROS) during hemodialysis, which contribute to DIOS.

Patients on maintenance hemodialysis suffer from increased oxidative stress promoting arteriosclerosis, the major cause for the excess mortality of this patient population. Excess oxygen attack the low density lipoproteins which leads to formation of plaque in the arteries leading to heart attack. Dialysis membranes may cause free oxidative radicals when contacting the patients' blood, which further deteriorates oxidative stress. The polymeric membrane coated with flavone of the present disclosure can reduces oxidative stress produced by dialysis membranes, which can be measured by different parameters in blood, such as the generation of peroxides and the oxidative burst.

The anti-oxidant properties of the polymeric membrane coated with stilbenoid or flavone can be understood by considering the mechanism of formation of oxygen radicals. Upon activation of the cells, the membrane-bound nicotinamide adenine dinucleotide phosphate (NADPH) and cytosolic components of the enzyme assemble in the membrane and form the active enzyme. NADPH oxidase catalyzes the reduction of O₂ to superoxide anion (O₂^{·-}), which then rapidly dismutates to hydrogen peroxide (H₂O₂). This chain of events is referred to as an electron transport chain Subsequently, H₂O₂ may be converted by the enzyme myeloperoxidase into highly reactive compounds such as hypochlorous acid (HOCl). Therefore, for a neutrophil to undergo oxidative burst, a functionally intact NADPH oxidase may be crucial. In this case, genistein successfully inhibited the expression of NADPH, which is the first step of the electron transport chain to form superoxide anion and subsequent dismutation to H₂O₂.

The polymeric membrane of the present discourse have been found to reduce serum levels of certain cytokines as well as promoting a reduction in reactive oxygen species (ROS), which are known to play an important role in mutagenesis, carcinogenesis and particularly in tumor promotion. Genistein can inhibit both the priming events necessary for high level ROS production. In some embodiments, the polymeric membrane of the present disclosure can reduce Oxidative Burst (i.e., generation of ROS) by more than 50%, more than 60%, or more than 70% compared to the membrane without flavone coating, which is measured by the method described in the Example. In some embodiments, the polymeric membrane of the present disclosure can H₂O₂ by more than 20%, or more than 25%, more than 30%, more than 35%, more than 40%, more than 45%, or more than 50% compared to the membrane without flavone coating, which is measured by the method described in the Example.

The polymeric membrane of the present discourse have been found to be capable of effectively retaining blood cells, such as white blood cell, red blood cell, platelet, etc., which are important for blood function. In some embodiments, the polymeric membrane of the present disclosure can retain more than 90%, 95%, 98%, 99%, or100% white blood cell or red blood cell measured by the method described in the Example. In some embodiments, the polymeric membrane of the present disclosure can retain more than 70%, 75%, 80%, 85%, or 90% platelet, measured by the method described in the Example. In some embodiments, the polymeric membrane of the present disclosure can increase the retention of platelet by more than 50%, 60%, 70%, 80%, 90% or 100% compared to the membrane without flavone coating.

The polymeric membrane of the present discourse can reduce the concentrations of Thrombin-Antithrombin Complex (TAT) to reduce the effects of dialysis membranes on the coagulation system and cell activation, without being toxic or activating the platelet adhesion process. Thrombin-Antithrombin Complex (TAT) increase significantly during the coagulation process, which can lead to activation of platelet. In some embodiments, the polymeric membrane of the present disclosure can decrease TAT levels in plasma by more than 10%, 20%, or 30% % compared to the membrane without flavone coating.

The polymeric membrane of the present discourse can be suitable for use in applications in the field of filtration. Due to the unique combination of properties of the polymeric membrane as described herein, preferably obtained from the method as described herein, the present disclosure further provides a use of the membranes as described herein for filtration of liquids, for example, microfiltration or ultrafiltration. “Microfiltration” and “ultrafiltration” have the meaning common in the art. Preferably, the use as described herein comprises clarification and/or purification of liquid media, in particular aqueous liquids. The polymeric membrane of the present discourse can be used for multiple extracorporeal blood purification procedures including dialysis.

The following working examples are intended to be illustrative of the present disclosure and not limiting.

### EXAMPLES

Objects and advantages of this invention are further illustrated by the following examples, but the particular materials and amounts thereof recited in these examples, as well as other conditions and details, should not be construed to unduly limit this invention.

Materials and Test Methods

Genistein was obtained from Herb-key (China Shaanxi NHK Technology), Shaanxi, China Resveratrol (product no. R5010) was obtained from the Sigma-Aldrich Company, St. Louis, MO.

White blood cell counts (WBC), red blood cell counts (RBC), and platelet counts (PC) were determined using an ABX Pentra 60 cell counter (Axon Lab AG, Reichenbach, Germany).

Total lipid peroxide levels in plasma samples were determined using chromogenic assay kits (obtained from Immundiagnostik AG, Bensheim Germany) according to the manufacturer's instructions.

Complement Component 5a (C5a) levels in plasma samples were determined using ELISA assay kits (obtained from DRG Instrument GmbH, Marburg, Germany) according to the manufacturer's instructions.

Thrombin-Antithrombin Complex (TAT) levels in plasma samples were determined using ELISA assay kits (obtained from Siemens Healthcare Diagnostics, Marburg, Germany) according to the manufacturer's instructions.

Oxidative burst activity of blood samples was determined by flow-cytometry using a FACS VERSE flow cytometer (Becton Dickinson GmbH, Heidelberg, Germany). Blood samples were incubated for 10 minutes at 37° C. with dihydrorhodamine 123 (DHR123) non-fluorescent dye (DHR 123 taken up by neutrophils in the sample). Next, blood cells were stimulated with and without a formyl-peptide (N-formyl Nle-Leu-Phe-Nle-Try-Lys) for 15 minutes at 37° C. and then red blood cells were lysed with BD PHARM LYSE lysing solution (Becton Dickinson GmbH). Upon generation of reactive oxygen species, DHR was oxidized to the fluorescent dye rhodamine In the assay, oxidative burst activity was proportional to the intracellular fluorescence intensity (relative fluorescence units (RFU)) of rhodamine measured by the flow-cytometer. In each sample 5,000 neutrophils were acquired within a gate of forward scatter (FSC) vs. side scatter (SSC). FSC and SSC were analyzed on a linear scale and the fluorescence data on a biexponential scale. Data acquisition and analysis was conducted using FACSUITE Software (version 1.05, Becton Dickinson GmbH). Lipopolysaccharide (100 ng/mL, from R 595 of S. minnesota) primed blood samples stimulated with formyl-peptide served as positive controls. Oxidative burst activity was expressed as the geometric mean of the fluorescence intensity (RFU) of rhodamine and calculated from the difference of the geometric mean of samples incubated with and without formyl-peptide.

Hemolysis was measured by spectrophotometry (UV1650PC spectrophotometer, Shimadzu Deutschland GmbH, Duisburg, Germany) at three wavelengths (OD_{380 nm}, OD_{415 nm}, OD_{450 nm}) to correct for background according to the reference of Herboe, M, Scandinavian Journal of Clinical and Lab Investigation, 1959, 11, pages 66-70. Equation A was used to calculate plasma free hemoglobin [fHb] (g/dL). To reflect hemolysis, fHb at the end of the experiment was set in relation to total hemoglobin at baseline. Equation A:

fHb=[(168*A415 nm)−(84*A380 nm)−(84*A450 nm)]*(DF/11); where ‘DF’ is the plasma dilution factor

### Example 1. Preparation of Dialyzer Modules Containing Membranes that were Coated with Genistein

A coating solution of genistein (0.4 weight %) in ethanol was prepared and added to a 5 L pressurizable vessel. A PUREMA polyethersulfone, hollow fiber, capillary membrane Type H (inner diameter 200 micrometers, wall thickness 30 micrometers, active surface area 1.1 m², obtained from the 3M Company, St. Paul, MN) was inserted in a dialyzer module. The dialyzer module was tube shaped with an open connector element at each end of the tube. The two side ports located proximate the upper and lower ends of the module were closed with clamps The dialyzer module was mounted in a vertical orientation with the lower connector of the module connected to the valve port of the pressurizable vessel using PTFE (polytetrafluoroethylene) tubing. The vessel was pressurized (0.4 bar) and the valve was opened causing the coating solution to flow into the lumen portion of the membrane. The valve was closed when the lumen portion was completely filled and coating solution began to exit from the open upper connector. The coating solution was maintained in the dialyzer module for 10 minutes and then the tubing was removed from the lower connector to allow the coating solution to drain from the dialyzer module. During the process, coating solution was observed to penetrate the capillary membrane wall filling part of the extra-capillary volume. After draining of the coating solution, a nitrogen gas source was attached at the lower connector and a gentle stream of nitrogen was passed through the membrane to evaporate residual ethanol.

### Comparative Example A. Preparation of Dialyzer Modules Containing Membranes that were Not Coated with Genistein

The same procedure as described in Example 1 was followed with the exception that a different coating solution was used. The coating solution was ethanol with no other additives.

### Example 2. Analysis of Human Blood

Dialyzer modules prepared according to Example 1 and Comparative Example A were analyzed using freshly donated human blood samples (pooled from 2-3 donors). The same pool of heparinized blood (3.5 IU/mL standard heparin, #H3149, Sigma-Aldrich, Steinheim, Germany) was used as the source of blood for all experiments. The two side ports of each module were closed with clamps Each dialyzer module was mounted in a vertical orientation and an aqueous saline solution (1 L, concentration of NaCl 0.9%) was recirculated through the module at 250 mL/minute for 30 minutes. The saline flowed through the module in the direction from the lower connector to the upper connector. Next, a second liter of NaCl solution (0.9%) was pumped through the module in a single pass (60 mL/minute) with the liquid flowing in the direction from the lower connector to upper connector and exiting from the module through the upper connector. An aliquot (240 mL) from the pool of heparinized blood was then recirculated through the module at 250 mL/minute for 180 minutes. The blood was maintained at 37° C. during the recirculation step and the flow of blood through the module was in the same direction as with the previous saline recirculation steps. Samples of blood were collected both before (t=0) and after the recirculation step (t=180) and subsequently analyzed. The results are reported in Table 1. For WBC, RBC, and PC, the counts obtained after the recirculation step (at t=180) were compared to the corresponding baseline counts obtained before the recirculation step (at t=0) and then reported as the percent relative to baseline. TAT, C5a, total lipid peroxides, and oxidative burst activity were measured using blood samples obtained after the recirculation step (t=180).

For the module of Example 1, the results are reported from a single experiment. For the module of Comparative Example A, the results are reported as the average values (with standard deviation) from experiments with three separate modules (n=3). A separate aliquot of heparinized blood from the blood pool was used with each module tested.

TABLE 1
Human Blood Analysis Using the Procedure of Example 2

Dialyzer Module

| | Example 1 | Comparative Example A |
|---|---|---|
| | (n = 1) | (n = 3) |
| WBC (%) | 101 | 97 ± 1 |
| RBC (%) | 102 | 101 ± 2 |
| PC (%) | 91 | 56 ± 11 |
| TAT (ng/mL) | 10.4 | 24.5 ± 5.1 |
| C5a (ng/ml) | 0.6 | 0.8 ± 0.4 |
| Hemolysis (%) | 0.32 | 0.31 ± 0.08 |
| Total Lipid Peroxides | 204 | 423 ± 147 |
| (micromol/L) | | |
| Oxidative Burst | 280 | 967 ± 666 |
| Activity (RFU) | | |

Example 3.

A sheet of 3M MICROPES Type 1F PH polethersulfone membrane (110 micrometers thick, obtained from the 3M Company) was immersed in a solution of resveratrol (0.8 weight %) in ethanol for 5 minutes at room temperature. The membrane was removed from the solution and dried at room temperature overnight.

### Comparative Example B.

A sheet of 3M MICROPES Type 1F PH polyethersulfone membrane (110 micrometers thick) was immersed in ethanol for 5 minutes at room temperature. The membrane was removed from the solution and dried at room temperature overnight.

### Example 4.

Samples (4.5 cm diameter) were punched from membranes prepared according to Example 3 and Comparative Example B. Each sample was placed in a Petri dish containing 10 mL of a saline solution (concentration of NaCl 0.9%). The dish was shaken on an orbital shaker for 40 minutes at 70 rpm 10 (revolutions per minute). The saline solution was then removed from the dish, replaced with 10 mL of fresh saline solution, and the dish was shaken for 1 minute. Next, the saline solution was removed from the dish, replaced with 8 mL of heparinized human blood (3.5 IU/mL standard heparin, #H3149, Sigma-Aldrich) and shaken (70 rpm) for 3 hours at 37° C. Each blood sample was tested for total lipid peroxides and oxidative burst activity. The results are reported in Table 2 as the average value (with standard deviation) from experiments with three separate membrane samples (n=3).

TABLE 2
Human Blood Analysis Using the Procedure of Example 4

Membrane Sample

| | Example 3 | Comparative Example B |
|---|---|---|
| Total Lipid Peroxides | 4 ± 3 | 527 ± 183 |
| (micromol/L) | | |
| Oxidative Burst | 1075 ± 1196 | 3723 ± 4306 |
| Activity (RFU) | | |

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A membrane comprising:
a polymeric membrane made from a polymer selected from an aromatic sulfone polymer, polyamide, cellulose, cellulose acetate, polymethylmethacrylate, polyvinylalcohol, and polyacrylnitril, wherein the polymeric membrane has a major surface; and
a stilbenoid, isoflavone or flavone coated on the major surface of the polymeric membrane.

2. The polymeric membrane of claim 1, wherein the stilbenoid, isoflavone or flavone covers more than 75% major surface of the polymeric membrane.

3. The polymeric membrane of claim 1, wherein the stilbenoid, isoflavone or flavone covers more than 80% major surface of the polymeric membrane.

4. The polymeric membrane of claim 1, wherein the polymeric membrane is a hollow fiber membrane comprises an inner surface facing towards its lumen, an outer surface facing outwards and an intermediate wall having a wall thickness, and wherein the major surface is the inner surface.

5. The polymeric membrane of claim 4, wherein the intermediate wall comprises a plurality of pores and the stilbenoid, isoflavone or flavone coated on a surface of at least some of plurality of pores.

6. The polymeric membrane of claim 1, wherein the aromatic sulfone polymer comprises a polyethersulfone.

7. The polymeric membrane of claim 1, wherein the isoflavone comprises a hydroxyisoflavone.

8. The polymeric membrane of claim 1, wherein the isoflavone is selected from the group consisting of genistein, daidzein, glycitein, prunetin, biochanin A, orobol, santal, pratensein, formononetin, and glucosides, 13-glycosides, and alkoxy substituted derivatives thereof, and combinations thereof.

9. The polymeric membrane of claim 1, wherein the isoflavone is selected from the group consisting of genistein, daidzein, and combinations thereof.

10. A method, the method comprising:
forming a polymeric membrane from an aromatic sulfone polymer; and
coating a stilbenoid, isoflavone or flavone to the hollow fiber membrane.

11. The method of claim 10, wherein the coating step comprises dissolving the stilbenoid, isoflavone or flavone in a solvent to form a coating solution.

12. The method of claim 11, wherein the solvent is selected from the group consisting of ethanol and isopropanol.

13. The method of claim 10, wherein the coating step comprises immersing the polymeric membrane with the coating solution.

14. The method of claim 10, further comprising draining the coating solution from polymeric membrane after the polymeric membrane is immersed with the coating solution.

15. The method of claim 10, further comprising drying the polymeric membrane after draining.

16. The method of claim 10, wherein the isoflavone is selected from the group consisting of genistein, daidzein, and combinations thereof.

17. The method of claim 10, wherein the polymeric membrane is a hollow fiber membrane having lumens.

18. The method of claim 17, draining the coating solution comprises draining the coating solution from of the hollow fiber membrane after the lumens of the hollow fiber membrane are filled with the coating solution.

19. The method of claim 10, wherein the coating step comprises flowing the coating solution into lumens of the polymeric membrane.

20. A use of the polymeric membrane of claim 1 for filtration of liquids.
